SEQUENCE LISTING (14 sequences)

SEQ ID NO: 1 - DNA, 118 bp, Artificial Sequence
  Single strand DNA aptamer to bisphenol A, screened by in vitro selection method.
taatacgactcactatagggaattcgtcgacggatcctggtcgttggtcgttcgcgtttctggattttttatttctggggttcagttcttttttgtctacaggtcgacgcatgcgccg

SEQ ID NO: 2 - DNA, 118 bp, Artificial Sequence
  Single strand DNA aptamer to bisphenol A, screened by in vitro selection method.
taatacgactcactatagggaattcgtcgacggatcccaagggccgagcgtacctggtttgctcgttttttgtcgaatttttggcgccttatatttctgcaggtcgacgcatgcgccg

SEQ ID NO: 3 - DNA, 113 bp, Artificial Sequence
  Single strand DNA aptamer to bisphenol A, screened by in vitro selection method.
taatacgactcactatagggaattcgtcgacggatccttgtgtaggatttaggggatattttttatcttattctttgacgcgcaaattctactgcaggtcgacgcatgcgccg

SEQ ID NO: 4 - DNA, 117 bp, Artificial Sequence
  Single strand DNA aptamer to bisphenol A, screened by in vitro selection method.
taatacgactcactatagggaattcgtcgacggatccaaagtggcctgcaatccctcggtattttagtcttttgtttttgctgtattcctttcatctgcaggtcgacgcatgcgccg

SEQ ID NO: 5 - DNA, 116 bp, Artificial Sequence
  Single strand DNA aptamer to bisphenol A, screened by in vitro selection method.
taatacgactcactatagggaattcgtcgacggatccggcctgtatggcatgctgcgctattttcactcacatgttctttttattcttttggttctgcaggtcgacgcatgcgccg

SEQ ID NO: 6 - DNA, 118 bp, Artificial Sequence
  Single strand DNA aptamer to bisphenol A, screened by in vitro selection method.
taatacgactcactatagggaattcgtcgacggatccggtccattcagcctctattaatcccctagtctactacttttctcgtctggttttctttcctgcaggtcgacgcatgcgccg

SEQ ID NO: 7 - DNA, 117 bp, Artificial Sequence
  Single strand DNA aptamer to bisphenol A, screened by in vitro selection method.
taatacgactcactatagggaattcgtcgacggatccggtgaatcagtctcttatcattttttcgattcttagccggattaacaattctttactcctgcaggtcgacgcatgcgccg

SEQ ID NO: 8 - DNA, 109 bp, Artificial Sequence
  Single strand DNA aptamer to bisphenol A, screened by in vitro selection method.
taatacgactcactatagggaattcgtcgacggatccggatgtggtctttatttttgtatcctcggcatcctcctccggcccgttccctgcaggtcgacgcatgcgccg

SEQ ID NO: 9 - DNA, 118 bp, Artificial Sequence
  Single strand DNA aptamer to bisphenol A, screened by in vitro selection method.
taatacgactcactatagggaattcgtcgacggatcctctcgaatattattttcccgtaaactcttcggagggtagccatttttcctcgttgagtactgcaggtcgacgcatgcgccg

SEQ ID NO: 10 - DNA, 108 bp, Artificial Sequence
  Single strand DNA aptamer to bisphenol A, screened by in vitro selection method.
taatacgactcactatagggaattcgtcgacggatccgatatttagggcgcgtccggcaccttttattttttcttgattggtttttctgcaggtcgacgcatgcgccg

SEQ ID NO: 11 - DNA, 119 bp, Artificial Sequence
  Single strand DNA aptamer to bisphenol A, screened by in vitro selection method.
taatacgactcactatagggaattcgtcgacggatccgattgttgcggagttctgttttctttggcggttattttttctatttcttagcaggtcgacctgcaggtcgacgcatgcgccg

SEQ ID NO: 12 - DNA, 103 bp, Artificial Sequence
  unsure (23)..(81): A,G,C or T
tagggaattcgtcgacggatccnnnnnnnnnnnnnnnnnnnnnnnnnnnnnnnnnnnnnnnnnnnnnnnnnnnnnnnnnnnctgcaggtcgacgcatgcgccg

SEQ ID NO: 13 - DNA, 35 bp, Artificial Sequence
  Oligo-DNA designed to act as PCR primer (sense) for amplification of DNA sequence of SEQ ID NO 12.
taatacgactcactatagggaattcgtcgacggat

SEQ ID NO: 14 - DNA, 19 bp, Artificial Sequence
  Oligo-DNA designed to act as PCR primer (antisense) for amplification of DNA sequence of SEQ ID NO 12.
cggcgcatgcgtcgacctg

(19) United States
(12) Patent Application Publication
(10) Pub. No.: US 2005/0282226 A1
(43) Pub. Date: Dec. 22, 2005

(54) Aptamer capable of specifically adsorbing to bisphenol a and method for obtaining the aptamer

(75) Inventors: Keisaku Okada, Ibaraki-shi (JP); Shuji Senda, Ibaraki-shi (JP); Akio Kobayashi, Toyonaka-shi (JP); Eiichiro Fukusaki, Suita-shi (JP); Itaru Yanagihara, Sakai-shi (JP); Tsuyoshi Nakanishi, Yao-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Nitto Denko Corporation, Ibaraki-shi (JP)

(21) Appl. No.: 11/214,437

(22) Filed: Aug. 29, 2005

Related U.S. Application Data
(63) Continuation of application No. 10/189,954, filed on Jul. 3, 2002.

(30) Foreign Application Priority Data
Jul. 4, 2001 (JP) 203862/2001

(51) Int. Cl. C12Q 1/68; C07H 21/04

(52) U.S. Cl. 435/6; 536/25.4

(57) ABSTRACT

The present invention provides an aptamer capable of specifically adsorbing to bisphenol A suspected to be an endocrine disrupter as a target molecule, a method for obtaining an aptamer capable of specifically adsorbing to bisphenol A by an in vitro selection method utilizing affinity chromatography using a carrier immobilizing bisphenol A, particularly, a method including use of an antagonistic elution buffer containing an amphiprotic organic solvent for elution by the affinity chromatography, and a single-strand nucleic acid molecule which is an aptamer obtained thereby.

### TECHNICAL FIELD OF THE INVENTION

The present invention relates to a single-strand nucleic acid molecule (aptamer) capable of specifically adsorbing to bisphenol A and a method for obtaining the aptamer.

### BACKGROUND OF THE INVENTION

Certain kinds of chemical substances are known to give an adverse influence on reproduction of human and wild animals when they are released in the environment. Since these chemical substances show a similar effect on hormones in the body and are considered to disturb the endocrine mechanism of wild animals and human, they are called “endocrine disrupters”, popularly referred to as an “environmental hormone”. Of the chemical substances suspected to be the endocrine disrupters, bisphenol A is a representative one being used in our immediate circles.

Bisphenol A is a symmetric divalent phenol produced by a condensation reaction of phenol and acetone in the presence of an acidic catalyst, and is widely used as a starting material of general purpose plastic such as polycarbonate resin, epoxy resin and the like. The polycarbonate resin is used for, for example, feeding bottles, tableware for school lunch, compact disc (CD), cellular phone, OA equipment and the like. The epoxy resin is used for, for example, a corrosion inhibitory coating for cans, water pipes and the like, adhesive, wiring substrate for electric appliances and the like.

The above-mentioned resin products include unreacted bisphenol A, and bisphenol A elutes out in the environment depending on the manner of use of the resin products. The adverse effect of bisphenol A in the level of elution from resin products on the body is unknown at the moment, but the development of a high affinity ligand affording selective recognition of bisphenol A from among the organic compounds having various low molecular weights, as a tool for the study of bisphenol A suspected of correlation with endocrine disrupters, is extremely important.

With the advance in the evolutionary molecular engineering in recent years, a technique for screening a nucleic acid molecule having high affinity for a target molecule (e.g., protein etc.), or an aptamer, from a random oligonucleotide library has been developed. This method is called an in vitro selection method, SELEX (Systematic evolution of ligands by exponential enrichment) and the like, and there are many reports on the preparation of high affinity ligand using this method, which is quicker and easier than the preparation of an antibody (e.g., Nature, 355: 564 (1992), WO92/14843, EP 0533838, U.S. Pat. No. 5,780,449 etc.).

However, obtaining an affinity ligand of a low molecular weight organic compound is considered to be generally difficult, because the compound has a smaller molecular weight and epitope is limited for recognition. As regards bisphenol A, too, a high affinity aptamer capable of selective recognition thereof has not been obtained.

### SUMMARY OF THE INVENTION

The present invention aims at providing a method for obtaining a novel affinity ligand or aptamer capable of specifically recognizing and adsorbing to bisphenol A suspected to be an endocrine disrupter, as a target molecule, and the aptamer.

Accordingly, the present invention provides the following.

(1) A method for obtaining an aptamer capable of specifically adsorbing to bisphenol A by an in vitro selection method utilizing affinity chromatography using a carrier immobilizing bisphenol A, which comprises using an antagonistic elution buffer containing an amphiprotic organic solvent for elution by the above-mentioned affinity chromatography.

(2) The method of the above-mentioned (1), wherein the above-mentioned affinity chromatography comprises a washing treatment using a washing buffer containing an amphiprotic organic solvent.

(3) The method of the above-mentioned (1), wherein the carrier immobilizing bisphenol A is packed in an affinity column.

(4) The method of the above-mentioned (1), wherein the antagonistic elution buffer containing an amphiprotic organic solvent comprises 2%-50% of the amphiprotic organic solvent.

(5) The method of the above-mentioned (2), wherein the washing buffer containing an amphiprotic organic solvent comprises 2%-50% of the amphiprotic organic solvent.

(6) The method of the above-mentioned (1) or (2), wherein the amphiprotic organic solvent is at least one of dimethyl sulfoxide, dioxane, N,N-dimethylformamide, tetrahydrofuran and ethanol.

(7) A single-strand nucleic acid molecule which is an aptamer obtained by the method of the above-mentioned (1).

(8) A method for obtaining an aptamer capable of specifically adsorbing to bisphenol A by an in vitro selection method utilizing affinity chromatography using an affinity column immobilizing bisphenol A, which comprises using an antagonistic elution buffer containing 2%-50% of an amphiprotic organic solvent for elution by the above-mentioned affinity chromatography.

(9) The method of the above-mentioned (8), wherein the above-mentioned affinity chromatography comprises a washing treatment using a washing buffer containing 2%-50% of an amphiprotic organic solvent.

(10) The method of the above-mentioned (8) or (9), wherein the above-mentioned amphiprotic organic solvent is at least one of dimethyl sulfoxide, dioxane, N,N-dimethylformamide, tetrahydrofuran and ethanol.

(11) A single-strand nucleic acid molecule which is an aptamer obtained by the method of any of the above-mentioned (8)-(10).

(12) A single-strand nucleic acid molecule, which is an aptamer capable of specifically adsorbing to bisphenol A, and which comprises any of the following base sequences (a)-(l):

(a) a base sequence consisting of 38^{th}-96^{th} nucleotides depicted in SEQ ID NO: 1, provided that when the nucleic acid molecule is an RNA, T in the sequence is U,
(b) a base sequence consisting of 38^{th}-96^{th} nucleotides depicted in SEQ ID NO: 2, provided that when the nucleic acid molecule is an RNA, T in the sequence is U,
(c) a base sequence consisting of 38^{th}-91^{st} nucleotides depicted in SEQ ID NO: 3, provided that when the nucleic acid molecule is an RNA, T in the sequence is U,
(d) a base sequence consisting of 38^{th}-95^{th} nucleotides depicted in SEQ ID NO: 4, provided that when the nucleic acid molecule is an RNA, T in the sequence is U,
(e) a base sequence consisting of 38^{th}-94^{th} nucleotides depicted in SEQ ID NO: 5, provided that when the nucleic acid molecule is an RNA, T in the sequence is U,
(f) a base sequence consisting of 38^{th}-96^{th} nucleotides depicted in SEQ ID NO: 6, provided that when the nucleic acid molecule is an RNA, T in the sequence is U,
(g) a base sequence consisting of 38^{th}-95^{th} nucleotides depicted in SEQ ID NO: 7, provided that when the nucleic acid molecule is an RNA, T in the sequence is U,
(h) a base sequence consisting of 38^{th}-87^{th} nucleotides depicted in SEQ ID NO: 8, provided that when the nucleic acid molecule is an RNA, T in the sequence is U,
(i) a base sequence consisting of 38^{th}-96^{th} nucleotides depicted in SEQ ID NO: 9, provided that when the nucleic acid molecule is an RNA, T in the sequence is U,
(j) a base sequence consisting of 38^{th}-86^{th} nucleotides depicted in SEQ ID NO: 10, provided that when the nucleic acid molecule is an RNA, T in the sequence is U,
(k) a base sequence consisting of 38^{th}-97^{th} nucleotides depicted in SEQ ID NO: 11, provided that when the nucleic acid molecule is an RNA, T in the sequence is U,
(l) any of the above-mentioned base sequences (a) to (k), wherein 1 to several nucleotides have been deleted, substituted, inserted or added.
   (13) The single-strand nucleic acid molecule of the above-mentioned (12), wherein the nucleic acid is a DNA.

### BRIEF DESCRIPTION OF THE DRAWINGS

FIG. 1 is a flow chart showing a series of steps of a preferable method of the present invention for obtaining an aptamer capable of specifically adsorbing to bisphenol A.

### DETAILED DESCRIPTION OF THE INVENTION

In the present specification, by the “in vitro selection method” is meant a method for obtaining a nucleic acid molecule having a particular function by repeatedly performing a selection process including separation of a single strand oligonucleotide having a particular function (e.g., specific adsorption to a target substance) from a randomly synthesized single strand oligonucleotide library, amplification of the oligonucleotide, and separation of a single strand oligonucleotide having the above-mentioned particular function. When a nucleic acid molecule (aptamer) capable of specific adsorption to a particular target substance is to be obtained from a random oligonucleotide library, the above-mentioned nucleic acid molecule capable of adsorption is separated by, for example, affinity chromatography using an affinity column having a target substance immobilized thereon.

In the present specification, by the “aptamer” is meant a single-strand nucleic acid molecule capable of specific adsorption to a particular target substance. The aptamer in the present specification is not limited to those obtained by the above-mentioned in vitro selection method.

In the present specification, by the “affinity chromatography” is meant a separation method utilizing a specific interaction (affinity) that a biological substance shows. The separation means is not particularly limited, and various methods usually employed in the pertinent field are used. To be specific, affinity chromatography using an affinity column is exemplified. This method includes at least the steps of (i) applying a substance capable of specifically adsorbing to a target substance and/or a substance incapable of adsorbing to an affinity column packed with a carrier having a target substance immobilized thereon (hereinafter sometimes to be conveniently referred to as a target substance-immobilized affinity column), (ii) washing, after the application, the column with a washing buffer to separate the above-mentioned substance capable of adsorption from a substance incapable of adsorption (washing treatment), and (iii) weakening, after the washing treatment, the bonding force between a substance capable of adsorption and the target substance immobilized on the column, with an elution buffer to allow elution of the substance capable of adsorption (elution treatment). As the carrier used for immobilizing the target substance, those known to be used for affinity chromatography, particularly affinity column chromatography, are mentioned.

### EMBODIMENT OF THE INVENTION

The present invention is described in detail in the following.

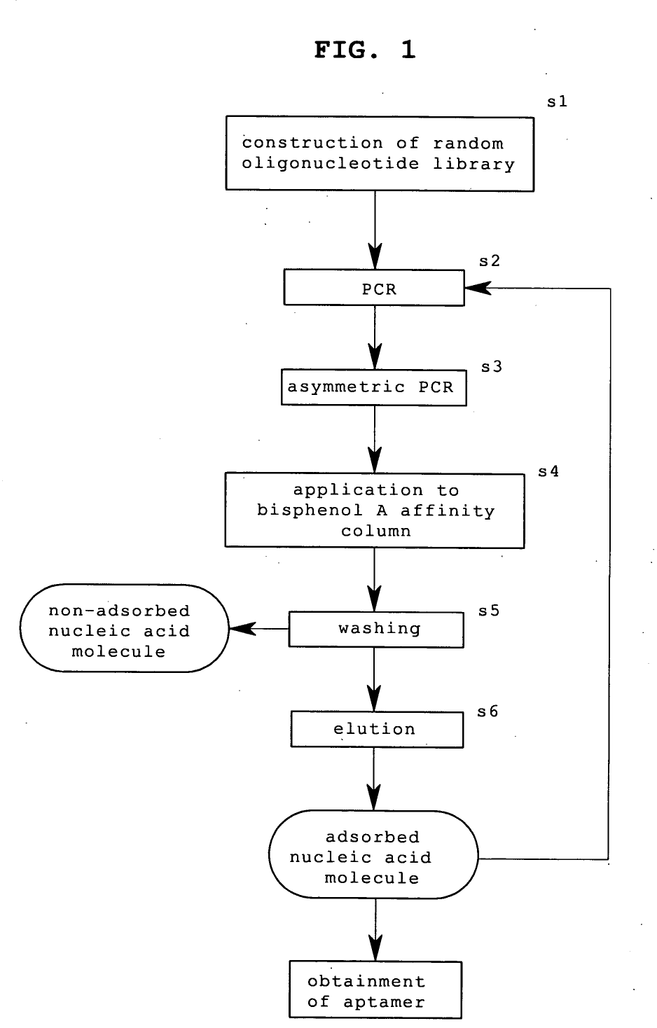

FIG. 1 is a flow chart showing a series of steps of a preferable method of the present invention for obtaining an aptamer capable of specifically adsorbing to bisphenol A.

In Step s1, a library of a single strand oligonucleotide (hereinafter sometimes to be also referred to as ssNt) containing a random region of a predetermined length of about 30 base-about 80 base is prepared using an automatic DNA/RNA synthesizer according to a conventional method. This library preferably contains 10¹³-10¹⁴ or more kinds of ssNt.

To facilitate PCR amplification in Step s2, s3 to be mentioned below, each ssNt is preferably designed to have a common priming site on both ends of the random region (i.e., a sequence homologous with sense primer on 5′-terminal and a sequence complementary to anti-sense primer on 3′-terminal), wherein “sense” and “anti-sense” primers are used to amplify the original ssNt and complementary chain thereof. Each of such priming sites is preferably designed to have a length of about 15 base-about 40 base, preferably about 15 base-about 30 base, and the corresponding PCR primers meet the general requirements of preferable primers.

When the aptamer after selection needs to be subcloned to a suitable vector, the priming site may contain a suitable restriction enzyme recognition site to facilitate the cloning. However, when amplification is performed by asymmetrical PCR such that a single strand oligonucleotide occupies the majority of the resulting amplified products, as in the embodiment shown in FIG. 1, the aptamer can be directly sequenced without subcloning.

In the subsequent Step s2, double stranded oligonucleotide (hereinafter sometimes to be referred to as dsNt) is amplified with the obtained ssNt library as a template and using sense and anti-sense primers corresponding to the priming sites on both ends of ssNt. Amplification of this dsNt can be performed by PCR according to a conventional method.

In the embodiment shown in FIG. 1, the above-mentioned dsNt library amplified by PCR is subjected to asymmetrical PCR using a sense primer alone in Step s3 to follow, whereby ssNt pool wherein sense strand (i.e., original ssNt) alone is amplified is prepared. This is because an aptamer is considered to have a specific adsorption capability to a target substance based on its structural and sequence characteristics that it is a single-strand nucleic acid molecule capable of forming a specific secondary structure, and therefore, it needs to be a single strand having an established given secondary structure before bisphenol A affinity chromatography in Steps s4-s6 below.

The ssNt amplified by asymmetrical PCR can be purified by agarose or polyacrylamide gel electrophoresis. Where desired, a PCR product may be subjected to ethanol precipitation for concentration prior to electrophoresis. A gel portion containing a band corresponding to the desired ssNt is recovered and ssNt is purified by a conventional method. ssNt is denatured at not lower than 90° C. prior to affinity chromatography and allowed to cool to ambient temperature to form a suitable secondary structure.

In the present invention, moreover, PCR may be performed by adding a sense primer in a great excess relative to the anti-sense primer (e.g., about 50-100:1), instead of PCR in the above-mentioned Step s2 and asymmetrical PCR in the above-mentioned Step s3, to prepare an ssNt pool wherein only sense strand is amplified.

The subsequent Step s4, Step s5 and Step s6 constitute a series of treatments of affinity chromatography using an affinity column in which bisphenol A is immobilized. To be specific, in Step s4, ssNt having a suitable secondary structure and obtained in Step s3 is applied to affinity column immobilizing bisphenol A, in Step s5, the affinity column is washed and the nucleic acid molecule that failed to adsorb to bisphenol A (hereinafter sometimes to be referred to as non-adsorbed nucleic acid molecule) is separated (washing treatment), and in Step s6, nucleic acid molecule that specifically adsorbed to bisphenol A (hereinafter sometimes to be referred to as adsorbed nucleic acid molecule) is eluted from the affinity column (elution treatment). According to the in vitro selection method of the present invention, the adsorbed nucleic acid molecule is separated from the non-adsorbed nucleic acid molecule utilizing the affinity chromatography using an affinity column immobilizing bisphenol A. For the affinity column to which ssNt is to be applied in Step s4, a column packed with beads and gel solid phase, on which bisphenol A has been immobilized in advance, may be used.

What is significant in the present invention is the use of a buffer containing an amphiprotic organic solvent for at least the elution in Step s6. Preferably, a buffer containing an amphiprotic organic solvent in a proportion of 2%-50%, preferably 5%-40%, is used for at least the elution in Step s6.

By the above-mentioned “amphiprotic organic solvent” is meant an active organic solvent having both acidity and basicity, wherein neither of them is remarkable. In the present invention, for example, dioxane, dimethyl sulfoxide (DMSO), N,N-dimethylformamide (DMF), tetrahydrofuran (THF), ethanol, methanol and the like are used. Of those mentioned above, an amphiprotic organic solvent selected from dioxane, DMSO, DMF, THF and ethanol is preferably used in view of solubility of bisphenol A and tolerance of column matrix and the like. The amphiprotic organic solvent may be a mixture of different kinds of amphiprotic organic solvents from among those mentioned above. As used herein, bisphenol A to be the target substance in the present invention is a chemical substance which has an aromatic ring and a symmetric structure and which is insoluble in water. While water is also an amphiprotic solvent, it is not used in the present invention. Instead, the above-mentioned amphiprotic organic solvent is used as an elution buffer capable of dissolving bisphenol A for antagonistic elution of a nucleic acid molecule specifically adsorbed to bisphenol A immobilized on an affinity column.

When the concentration of the amphiprotic organic solvent in the buffer for antagonistic elution in Step s6 is less than 2%, the selection efficiency of aptamer is degraded. When the concentration of the amphiprotic organic solvent in the buffer for antagonistic elution exceeds 50%, a column matrix is adversely affected as evidenced by precipitation of nucleic acid, denaturation of column resin and the like. The above-mentioned concentration refers to the volume proportion of the amphiprotic organic solvent (% by volume (v/v)) per volume of the buffer. When the amphiprotic organic solvent is in the form of a mixture, the above-mentioned concentration refers to the final concentration of the whole mixture.

In the present invention, moreover, a buffer containing an amphiprotic organic solvent, preferably a buffer containing an amphiprotic organic solvent in a proportion of 2%-50%, preferably 5%-30%, is preferably used in the washing treatment in Step s5. The amphiprotic organic solvent to be used for washing buffer include those exemplified for the aforementioned antagonistic elution buffers, from which an amphiprotic organic solvent selected from dioxane, DMSO, DMF, THF and ethanol is preferably used in view of the solubility of bisphenol A and tolerance of column matrix and the like. The amphiprotic organic solvent for washing buffer and the amphiprotic organic solvent in an antagonistic elution buffer may be the same or different.

When the concentration of the amphiprotic organic solvent in the washing buffer in Step s5 is less than 2%, the selection efficiency of aptamer is degraded. When the concentration of the amphiprotic organic solvent in the washing buffer exceeds 50%, a column matrix is adversely affected as evidenced by denaturation of column resin and the like.

The adsorbed nucleic acid molecule obtained by the above-mentioned Step s4-s6 is subjected to at least 5, preferably about 7-15, cycles of the above-mentioned PCR amplification (Step s2), asymmetrical PCR (Step s3) and affinity chromatography (Steps s4-s6) using an affinity column immobilizing bisphenol A, whereby the aptamer of the present invention can be obtained.

The obtained aptamer is made to be double stranded according to a conventional method and subcloned to a suitable vector using the restriction enzyme recognition site constructed in the priming site, by blunting the end, or by TA cloning method, after which its base sequence can be determined by the Maxam-Gilbert method or dideoxy method. Alternatively, the obtained single strand aptamer can be directly sequenced without subcloning.

According to the above-mentioned method of the present invention, an affinity ligand capable of specifically recognizing and adsorbing to bisphenol A as a target substance, which has been conventionally difficult to obtain, can be efficiently obtained.

The aptamer of the present invention capable of specifically adsorbing to bisphenol A is a single strand DNA or RNA, preferably a single strand DNA. While the length thereof is not particularly limited, it is preferably about 30 base-about 120 base.

The aptamer of the present invention substantially comprises, in the preferred embodiments, a base sequence consisting of 38^{th}-96^{th} nucleotides depicted in SEQ ID NO: 1, a base sequence consisting of 38^{th}-96^{th} nucleotides depicted in SEQ ID NO: 2, a base sequence consisting of 38^{th}-91^{st} nucleotides depicted in SEQ ID NO: 3, a base sequence consisting of 38^{th}-95^{th} nucleotides depicted in SEQ ID NO: 4, a base sequence consisting of 38^{th}-94^{th} nucleotides depicted in SEQ ID NO: 5, a base sequence consisting of 38^{th}-96^{th} nucleotides depicted in SEQ ID NO: 6, a base sequence consisting of 38^{th}-95^{th} nucleotides depicted in SEQ ID NO: 7, a base sequence consisting of 38^{th}-87^{th} nucleotides depicted in SEQ ID NO: 8, a base sequence consisting of 38^{th}-96^{th} nucleotides depicted in SEQ ID NO: 9, a base sequence consisting of 38^{th}-86^{th} nucleotides depicted in SEQ ID NO: 10, and a base sequence consisting of 38^{th}-97^{th} nucleotides depicted in SEQ ID NO: 11, wherein when the nucleic acid molecule is an RNA, T in the sequence is U. As used herein, by the “substantially comprises” is meant that any of the above-mentioned base sequences per se is included or any of the above-mentioned base sequences, wherein 1 to several nucleotides have been deleted, substituted, inserted or added and the bisphenol A specific adsorption capability is retained, is included.

A single-strand nucleic acid molecule (aptamer) substantially comprising the above-mentioned base sequence may not be prepared by the aforementioned method of the present invention, but may be prepared by any method, though preference is given to one prepared by the aforementioned method of the present invention.

### EXAMPLES

The present invention is explained in detail by referring to Examples. The Examples are mere exemplifications and do not limit the present invention in any way.

### Example 1

[1] Preparation of Amplified Single Strand DNA (ssDNA) Library

(1) Using an automatic DNA synthesizer, the following template DNA with 59 mer as a random region and a sense (P1) primer and an anti-sense (P2) primer were synthesized (Step s1).

(SEQ ID NO: 12)

Template:
5′-TAGGGAATTCGTCGACGGATCC-N59-CTGCAGGTCGACGCATGCGC
CG-3′

(SEQ ID NO: 13)

P1:
5′-TAATACGACTCACTATAGGGAATTCGTCGACGGAT-3′

(SEQ ID NO: 14)

P2:
5′-CGGCGCATGCGTCGACCTG-3′

(2) The above-mentioned template DNA was amplified by PCR using P1 and P2 primers (Step s2). The reaction mixture composition and reaction conditions were as follows.

Reaction Mixture Composition

| | | |
|---|---|---|
| distilled water | 73.5 | μl |
| 10 × PCR buffer* | 10 | μl |
| 20 mM dNTPs | 1 | μl |
| 10 μM P1 primer | 5 | μl |
| 10 μM P2 primer | 5 | μl |
| 1 μg/ml template DNA | 5 | μl |
| Ex Taq ™ DNA polymerase | 0.5 | μl (2.5 units) |

*10 × pCR buffer composition
100 mM Tris-HCl (pH 8.5)
500 mM KCl
20 mM MgCl₂

Reaction Conditions

| | |
|---|---|
| initial denaturation | 94° C., 1 min |
| denaturation | 94° C., 15 sec |
| annealing | 55° C., 15 sec 10 cycles |
| extension | 72° C., 15 sec |
| final extension | 72° C., 6 min |

(3) Using the above-mentioned PCR product as a template and P1 alone as a primer, asymmetrical PCR was performed (Step s3) to ultimately prepare 2 ml of PCR product (100 μl×20 tubes). The reaction mixture composition and reaction conditions were as follows.

Reaction Mixture Composition

| | | |
|---|---|---|
| distilled water | 78.5 | μl |
| 10 × pCR buffer | 10 | μl |
| 20 mM dNTPs | 1 | μl |
| 10 μM P1 primer | 5 | μl |
| 1 μg/ml template DNA | 5 | μl |
| Ex Taq ™ DNA polymerase | 0.5 | μl (2.5 units) |

Reaction Conditions

| | |
|---|---|
| initial denaturation | 94° C., 1 min |
| denaturation | 94° C., 15 sec |
| annealing | 55° C., 15 sec 40 cycles |
| extension | 72° C., 15 sec |
| final extension | 72° C., 6 min |

The PCR reaction mixture was dispensed by 400 μl to 5 microtubes. Thereto were added 10M ammonium acetate (80 μl) and 99.5% ethanol (1 ml) and gently mixed. The mixture was stood at −80° C. for 20 min. The mixture was centrifuged at 15,000 rpm for 15 min, rinsed with 70% ethanol and centrifuged at 15,000 rpm for 10 min. The precipitate was vacuum dried. Sterile distilled water (20 μl) was added and the mixture was vigorously mixed on Voltex to solve the precipitate. A gel loading buffer (20 μl, 95% formamide, 0.5 mM EDTA (pH 8.0), 0.025% STS, 0.025% xylene cyanol, 0.025% Bromophenol blue) was added and the mixture was thoroughly mixed on Voltex. The mixture was treated at 90° C. for 3 min to allow denaturation. The mixture was rapidly cooled on ice and subjected to electrophoresis (150 V, 50 min) on polyacrylamide gel. After immersing in ethidium bromide solution for about 5 min, the gel was washed with water and detected for a band on a transilluminator. The gel portion containing the objective band was cut out and ruptured. Elution buffer (800 μl, 0.5M ammonium acetate, 10 mM magnesium acetate, 1 mM EDTA (pH 8.0), 0.1% STS) was added and the mixture was shaken for 3 hr, and passed through a filter to recover the filtrate.

[2] Affinity Column Chromatography

(1) The DNA obtained in the above-mentioned [1] was precipitated with ethanol and, after vacuum drying, dissolved in distilled water (100 μl). A 2×binding buffer (100 μl, 200 mM Tris-HCl, 400 mM NaCl, 50 mM KCl, 20 mM MgCl₂ (pH 8.0), 10% dioxane (pH 8.0)) was added and thoroughly mixed and absorbance at 260 nm was measured. The DNA solution was treated at 90° C. for 5 min to allow denaturation, allowed to cool naturally and held. Formation of the secondary structure was confirmed by changes in absorbance.

(2) Bisphenol A was immobilized on a column as follows. First, bisphenol A and 4-bromo-n-butyric acid ethyl ester were coupled using potassium carbonate as a basic catalyst. The reaction conditions were stirring in dimethylformamide at room temperature for 4 hr. After the reaction, the reaction product was confirmed by thin-layer chromatography and extracted with ether. The reaction product was purified using Silica gel 60 (MERCK) to remove by-products. Then, the obtained compound was subjected to alkaline hydrolysis, whose reaction conditions were reflux for 2 hr in 95% ethanol in the presence of sodium hydroxide. The sample after the reaction was subjected to thin-layer chromatography and complete hydrolysis of the substrate was confirmed. Finally, synthesized bisphenol A derivative was immobilized on EAH Sepharose 4B (Amersham Pharmacia) by coupling reaction using carbodiimide. As a result of immobilization, 7.96 μmol of bisphenol A was bonded per 1 ml of the gel. The immobilized resin was filled in a 8 mm×5 mm column, washed with about 20-fold amount of water, and equilibrated with an about 20-fold amount of 1× binding buffer (100 mM Tris-HCl, 200 mM NaCl, 25 mM KCl, 10 mM MgCl₂, 5% dioxane (pH 8.0)), wherein the solution obtained then was used as a baseline.

(3) The DNA sample obtained in the above-mentioned (1) was applied to a column, and the eluate was received in a microtube upon opening the cock and applied again to the column (Step s4). This operation was repeated 3 times, and the column was left standing at room temperature for 30 min. A 1× binding buffer (5 ml, washing buffer) was poured and the cock was opened to fractionate in 6 microtubes by about 12 drops (about 650 μl) (Step s5). The cock was closed once, an elution buffer (100 mM Tris-HCl, 200 mM NaCl, 25 mM KCl, 10 mM MgCl₂, 30% dioxane (pH 8.0), 35.1 mM bisphenol A) (an antagonistic elution buffer) was poured thereon and the cock was opened. The eluate was received in a microtube and returned again to the column. This operation was repeated 3 times, whereby the buffer was substituted by an elution buffer. The cock was opened again to fractionate in 3 microtubes by about 12 drops (Step s6). The eluate was divided by 400 μl and glycogen (2 μl) was added thereto, followed by ethanol precipitation and vacuum drying. The precipitate was thoroughly dissolved in water (15 μl).

[3] Identification of Bisphenol A Specific DNA Aptamer

Each operation (Step s2-s6) of the above-mentioned [1] (2)-[2] (3) was repeated 12 times. The base sequence of 13 kinds of bisphenol A specific single strand DNA aptamers selected by the operation was determined by the dideoxy method.

The determined base sequence of random regions of each clones was as follows.

Clone 1:
5′-TGGTCGTTGGTCGTTCGCGTTTCTGGATTTTTTATTTCTGGGGTTCA
GTTCTTTTTTGT-3′

38^{th}-96^{th} nucleotides depicted in SEQ ID NO: 1

clone 2:
5′-CAAGGGCCGAGCGTACCTGGTTTGCTCGTTTTTTGTCGAATTTTTGG
CGCCTTATATTT-3′

38^{th}-96^{th} nucleotides depicted in SEQ ID NO: 2

clone 3:
5′-TTGTGTAGGATTTAGGGGATATTTTTTATCTTATTCTTTGACGCGCA
AATTCTA-3′

38^{th}-91^{st} nucleotides depicted in SEQ ID NO: 3

clone 4:
5′-AAAGTGGCCTGCAATCCCTCGGTATTTTAGTCTTTTGTTTTTGCTGT
ATTCCTTTCAT-3′

38^{th}-95^{th} nucleotides depicted in SEQ ID NO: 4

clone 5:
5′-GGCCTGTATGGCATGCTGCGCTATTTTCACTCACATGTTCTTTTTAT
TCTTTTGGTT-3′

38^{th}-94^{th} nucleotides depicted in SEQ ID NO: 5

clone 6:
5′-GGTCCATTCAGCCTCTATTAATCCCCTAGTCTACTACTTTTCTCGTC
TGGTTTTCTTTC-3′

38^{th}-96^{th} nucleotides depicted in SEQ ID NO: 6

clone 7:
5′-GGTGAATCAGTCTCTTATCATTTTTTCGATTCTTAGCCGGATTAACA
ATTCTTTACTC-3′

38^{th}-95^{th} nucleotides depicted in SEQ ID NO: 7

clone 8:
5′-GGATGTGGTCTTTATTTTTGTATCCTCGGCATCCTCCTCCGGCCCGT
TCC-3′

38^{th}-87^{th} nucleotides depicted in SEQ ID NO: 8

clone 4:
5′-TCTCGAATATTATTTTCCCGTAAACTCTTCGGAGGGTAGCCATTTTT
CCTCGTTGAGTA-3′

38^{th}-96^{th} nucleotides depicted in SEQ ID NO: 9,

clone 10: 5′-GATATTTAGGGCGCGTCCGGCACCTTTTATTTTTTCT
TGATTGGTTTTT-3′

38^{th}-86^{th} nucleotides depicted in SEQ ID NO: 10

clone 11: 5′-GATTGTTGCGGAGTTCTGTTTTCTTTGGCGGTTATTT
TTTCTATTTCTTAGCAGGTCGAC-3′

38^{th}-97^{th} nucleotides depicted in SEQ ID NO: 11

### Comparative Example 1

In the same manner as in Example 1 except that a buffer containing 100 mM Tris-HCl, 200 mM NaCl, 25 mM KCl, 10 mM MgCl₂, 0.5% dioxane (pH 8.0) and bisphenol A was used instead of the elution buffer (antagonistic elution buffer) used in the above-mentioned [2] (3), the experiment was performed. Since bisphenol A was not dissolved, recovery of aptamer from the column matrix was not attainable.

### Comparative Example 2

In the same manner as in Example 1 except that a buffer containing 100 mM Tris-HCl, 200 mM NaCl, 25 mM KCl, 10 mM MgCl₂, 60% dioxane (pH 8.0) and 35.1 mM bisphenol A was used instead of the elution buffer (antagonistic elution buffer) used in the above-mentioned [2] (3), the experiment was performed. Since nucleic acid molecules coagulated and precipitated during elution, elution from the column was not attainable and the aptamer could not be recovered.

As is clear from the foregoing explanation, the present invention affords a method for obtaining a novel affinity ligand or an aptamer capable of recognizing and specifically adsorbing to bisphenol A as a target substance. Inasmuch as the aptamer of the present invention can specifically recognize and adsorb to bisphenol A, it can be preferably used for the detection and quantitative determination of bisphenol A suspected to be an endocrine disrupter, study of the effect of bisphenol A to the body and the like, and is extremely useful.

This application is based on application No. 203862/2001 filed in Japan, the contents of which are incorporated hereinto by reference.

### Sequence Listing Free Text

SEQ ID NO: 1 single strand DNA aptamer to bisphenol A, screened by in vitro selection method
SEQ ID NO: 2 single strand DNA aptamer to bisphenol A, screened by in vitro selection method
SEQ ID NO: 3 single strand DNA aptamer to bisphenol A, screened by in vitro selection method
SEQ ID NO: 4 single strand DNA aptamer to bisphenol A, screened by in vitro selection method
SEQ ID NO: 5 single strand DNA aptamer to bisphenol A, screened by in vitro selection method
SEQ ID NO: 6 single strand DNA aptamer to bisphenol A, screened by in vitro selection method
SEQ ID NO: 7 single strand DNA aptamer to bisphenol A, screened by in vitro selection method
SEQ ID NO: 8 single strand DNA aptamer to bisphenol A, screened by in vitro selection method
SEQ ID NO: 9 single strand DNA aptamer to bisphenol A, screened by in vitro selection method
SEQ ID NO: 10 single strand DNA aptamer to bisphenol A, screened by in vitro selection method
SEQ ID NO: 11 single strand DNA aptamer to bisphenol A, screened by in vitro selection method
SEQ ID NO: 12 A, G, C or T single strand DNA containing 59mer random region flanked with PCR priming sites
SEQ ID NO: 13 oligo-DNA designed to act as a PCR primer (sense) for amplification of DNA sequence of SEQ ID NO: 12
SEQ ID NO: 14 oligo-DNA designed to act as a PCR primer (anti-sense) for amplification of DNA sequence of SEQ ID NO: 12

1. A method for obtaining an aptamer capable of specifically adsorbing to bisphenol A by an in vitro selection method utilizing affinity chromatography using a carrier immobilizing bisphenol A, which comprises using an antagonistic elution buffer containing an amphiprotic organic solvent for elution by the affinity chromatography.

2. The method of claim 1, wherein the carrier immobilizing bisphenol A is packed in an affinity column.

3. The method of claim 1, wherein the antagonistic elution buffer containing an amphiprotic organic solvent comprises 2%-50% of the amphiprotic organic solvent.

4. The method of claim 1, wherein the affinity chromatography comprises a washing treatment using a washing buffer containing an amphiprotic organic solvent.

5. The method of claim 4, wherein the washing buffer containing an amphiprotic organic solvent comprises 2%-50% of the amphiprotic organic solvent.

6. The method of claim 1, wherein the amphiprotic organic solvent in an antagonistic elution buffer is at least a solvent selected from the group consisting of dimethyl sulfoxide, dioxane, N,N-dimethylformamide, tetrahydrofuran and ethanol.

7. The method of claim 4, wherein the amphiprotic organic solvent in a washing buffer at least a solvent selected from the group consisting of dimethyl sulfoxide, dioxane, N,N-dimethylformamide, tetrahydrofuran and ethanol.

8. A single-strand nucleic acid molecule which is an aptamer obtained by the method of claim 1.

9. A method for obtaining an aptamer capable of specifically adsorbing to bisphenol A by an in vitro selection method utilizing affinity chromatography using an affinity column immobilizing bisphenol A, which comprises using an antagonistic elution buffer containing 2%-50% of an amphiprotic organic solvent for elution by the affinity chromatography.

10. The method of claim 9, wherein said affinity chromatography comprises a washing treatment using a washing buffer containing 2%-50% of an amphiprotic organic solvent.

11. The method of claim 9, wherein said amphiprotic organic solvent in an antagonistic elution buffer is at least a solvent selected from the group consisting of dimethyl sulfoxide, dioxane, N,N-dimethylformamide, tetrahydrofuran and ethanol.

12. The method of claim 10, wherein said amphiprotic organic solvent in a washing buffer is at least a solvent selected from the group consisting of dimethyl sulfoxide, dioxane, N,N-dimethylformamide, tetrahydrofuran and ethanol.

13. A single-strand nucleic acid molecule which is an aptamer obtained by the method of claim 9.

14. A single-strand nucleic acid molecule which is an aptamer obtained by the method of claim 10.

15. A single-strand nucleic acid molecule which is an aptamer obtained by the method of claim 11.

16. A single-strand nucleic acid molecule which is an aptamer obtained by the method of claim 12.

17. A single-strand nucleic acid molecule, which is an aptamer capable of specifically adsorbing to bisphenol A, and which comprises any of the following base sequences (a) to (l):
(a) a base sequence consisting of 38^{th}-96^{th} nucleotides depicted in SEQ ID NO: 1, provided that when the nucleic acid molecule is an RNA, T in the sequence is U,
(b) a base sequence consisting of 38^{th}-96^{th} nucleotides depicted in SEQ ID NO: 2, provided that when the nucleic acid molecule is an RNA, T in the sequence is U,
(c) a base sequence consisting of 38^{th}-91^{st} nucleotides depicted in SEQ ID NO: 3, provided that when the nucleic acid molecule is an RNA, T in the sequence is U,
(d) a base sequence consisting of 38^{th}-95^{th} nucleotides depicted in SEQ ID NO: 4, provided that when the nucleic acid molecule is an RNA, T in the sequence is U,
(e) a base sequence consisting of 38^{th}-94^{th} nucleotides depicted in SEQ ID NO: 5, provided that when the nucleic acid molecule is an RNA, T in the sequence is U,
(f) a base sequence consisting of 38^{th}-96^{th} nucleotides depicted in SEQ ID NO: 6, provided that when the nucleic acid molecule is an RNA, T in the sequence is U,
(g) a base sequence consisting of 38^{th}-95^{th} nucleotides depicted in SEQ ID NO: 7, provided that when the nucleic acid molecule is an RNA, T in the sequence is U,
(h) a base sequence consisting of 38^{th}-87^{th} nucleotides depicted in SEQ ID NO: 8, provided that when the nucleic acid molecule is an RNA, T in the sequence is U,
(i) a base sequence consisting of 38^{th}-96^{th} nucleotides depicted in SEQ ID NO: 9, provided that when the nucleic acid molecule is an RNA, T in the sequence is U,
(j) a base sequence consisting of 38^{th}-86^{th} nucleotides depicted in SEQ ID NO: 10, provided that when the nucleic acid molecule is an RNA, T in the sequence is U,
(k) a base sequence consisting of 38^{th}-97^{th} nucleotides depicted in SEQ ID NO: 11, provided that when the nucleic acid molecule is an RNA, T in the sequence is U,
(l) any of the base sequences (a) to (k), wherein 1 to several nucleotides have been deleted, substituted, inserted or added.

18. The single-strand nucleic acid molecule of claim 17, wherein the nucleic acid is a DNA.
